SEQUENCE LISTING (20 sequences)

SEQ ID NO: 1 - DNA, 23 bp, Homo sapiens
caggtgcagctactcgagtcggg

SEQ ID NO: 2 - DNA, 23 bp, Homo sapiens
caggtgcagctgctcgagtcggg

SEQ ID NO: 3 - DNA, 23 bp, Homo sapiens
caggtacagctcgagcagtcagg

SEQ ID NO: 4 - DNA, 27 bp, Homo sapiens
caggtacagctgctcgagtcaggtcca

SEQ ID NO: 5 - DNA, 24 bp, Homo sapiens
gacatcgagctcacccagtctcca

SEQ ID NO: 6 - DNA, 24 bp, Homo sapiens
gatattgagctcactcagtctcca

SEQ ID NO: 7 - DNA, 25 bp, Homo sapiens
  misc_feature (16)..(16): g or a
gaaattgagctcacgncagtctcca

SEQ ID NO: 8 - DNA, 20 bp, Homo sapiens
gaaatacctattgcctacgg

SEQ ID NO: 9 - DNA, 19 bp, Homo sapiens
gcgattgcagtggcactgg

SEQ ID NO: 10 - DNA, 354 bp, Homo sapiens
gaggtgcagctgctcgaggagtctgggggaggcttggtacagcctggagggtccctgagactctcctgtacagcctctagattcaccctcagtacttatgaaatgaactgggtccgccaggctccagggaaggggctggagtgggtttcatacattagtactagtggtggtatcatgtattatgcagactctgtgaggggccgattcaccatctccagagacaacgccaagaactcactgtatctccaaatgaacagcctgagagccgaggacacggctctttattactgtgcgaccgtgactttggatggcaacaacccttttgagaactggggccagggaaccctggtcacc

SEQ ID NO: 11 - protein, 118 aa, Homo sapiens
EVQLLEESGGGLVQPGGSLRLSCTASRFTLSTYEMNWVRQAPGKGLEWVSYISTSGGIMYYADSVRGRFTISRDNAKNSLYLQMNSLRAEDTALYYCATVTLDGNNPFENWGQGTLVT

SEQ ID NO: 12 - DNA, 340 bp, Homo sapiens
gcggccgagctcactcagtctccactctccctgtccgtcaccctcggacagccggcctccatagcctgcaagtctagtcatagcctcgcacgcagtgatggagtctctttgatgaattgggttcacctaaggccaggccggtctccaaggcgcctgatttatgacgtttctaagcgtgaaaatggggtcccagacagattcagcggcagtgggtcaggcactgacttcacactcgaaatcaacagggtggaggctgaagatgttggcctttattactgtctgcagtatgcacactggcctcacacttttggccaggggaccaagctggagatcaaacgaa

SEQ ID NO: 13 - protein, 113 aa, Homo sapiens
AAELTQSPLSLSVTLGQPASIACKSSHSLARSDGVSLMNWVHLRPGRSPRRLIYDVSKRENGVPDRFSGSGSGTDFTLEINRVEAEDVGLYYCLQYAHWPHTFGQGTKLEIKR

SEQ ID NO: 14 - DNA, 24 bp, Homo sapiens
caggtgcagctgctcgagtctggg

SEQ ID NO: 15 - DNA, 24 bp, Homo sapiens
gaggtgcagctcgaggagtctggg

SEQ ID NO: 16 - DNA, 23 bp, Homo sapiens
caggtgcagctactcgagtgggg

SEQ ID NO: 17 - DNA, 30 bp, Homo sapiens
gcatgtactagttttgtcacaagatttggg

SEQ ID NO: 18 - DNA, 24 bp, Homo sapiens
gacatcgagctcacccagtctcca

SEQ ID NO: 19 - DNA, 24 bp, Homo sapiens
gaaattgagctcacgcagtctcca

SEQ ID NO: 20 - DNA, 58 bp, Homo sapiens
gcgccgtctagaattaacactctcccctgttgaagctctttgtgacgggcgaactcag

(19) United States
(12) Patent Application Publication
(10) Pub. No.: US 2007/0003551 A1
(43) Pub. Date: Jan. 4, 2007

(54) Immunoglobulin

(76) Inventors: Nigel J. Lindsay, West Yorkshire (GB); Mohsen Hamidpour, West Yorkshire (GB); Lynda Partridge, South Yorkshire (GB)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/543,960

(22) PCT Filed: Feb. 5, 2004
(86) PCT No.: PCT/GB04/00444

§ 371 Date: Jun. 16, 2006

(30) Foreign Application Priority Data
Feb. 6, 2003 (GB) 0302699.4

(51) Int. Cl. A61K 39/395; G01N 33/567; C07K 16/40

(52) U.S. Cl. 424/146.1; 530/388.26; 435/7.2

(57) ABSTRACT

A recombinant human immunoglobulin exhibiting immuno-specificity for integrins is described. In addition, nucleotide and peptide sequences encoding the recombinant immunoglobulin are descrebed, and uses thereof.

The present invention relates to immunoglobulins, and particularly, although not exclusively, relates to recombinant immunoglobulins exhibiting specificity for integrins. The invention further relates to polynucleotide and peptide sequences encoding immunoglobulins, and uses thereof.

Integrins are a family of structurally, immunochemically, and functionally related heterodimeric molecules, consisting of α and β subunit chains. At present there are at least eight known β chains and at least seventeen known a chains which interact noncovalently in a restricted manner to form more than twenty family members.

Integrins, so named because they integrate from the extracellular matrix into the intracellular cytoskeleton, are receptors and are involved in cell-cell interactions such as cell adhesion, cell migration and coagulation responses. As a result, they make good candidates for therapeutic intervention by enabling the development of reagents that not only block platelet aggregation during blood clotting, but also act as anti-inflammatory reagents. This has been demonstrated by the existence of immunoglobulin reagents such as Abicimax which targets the receptor for fibrinogen, i.e. platelet integrin glycoprotein (GP) IIb/IIIa. Interaction between fibrinogen and GPIIb/IIIa is a crucial event in the initial aggregation of platelets, i.e. blood clotting. Platelet aggregation and adherence is the primary event in thrombosis which continues to be the major cause of death in heart attack and stroke.

The reagent Abicimax has been shown not only to react with integrin glycoprotein IIb/IIIa, but also the integrins MAC-1, LFA-1, VLA-4 and p150.95 which therefore also have potential as anti-inflammatory therapeutic targets.

However, a major disadvantage of Abicimax, is that it is derived from mouse monoclonal antibodies that have been ‘humanised’. Humanisation of a mouse monoclonal antibody involves replacing those parts of the mouse antibody molecule that are not involved with the direct interaction of the antibody with its target antigen, i.e. the framework regions of the antibody, with a human equivalent. This is a laborious procedure, and can result in the part of the antibody molecule that is involved with interaction with the target antigen, i.e. the Complementarity Determining Regions (CDRs) of the antibody, having a significant loss of affinity and therefore therapeutic potential.

The similarity between the integrins is such that the use of a small number of antibodies isolated from an immunoglobulin library could provide templates for generating a panel of different immunoglobulins that each react with a different integrin. Unfortunately, mouse/human hybrid immunoglobulins such as those described above are difficult to use as suitable templates.

An alternative approach to using mouse monoclonal antibodies is to produce a human immunoglobulin which would not require replacement of the framework regions of the antibody. Such an antibody could then be used as a template to develop a panel of antibodies showing reactivity to different integrins.

However, the major disadvantage with human immunoglobulins against human glycoprotein integrins, is that they are not easy to produce due to ethical issues surrounding immunising human patients against potentially pathogenic molecules. Hence, there is the need to produce a human immunoglobulin or monoclonal antibody having binding affinity with a human integrin without having to initially immunise a human patient.

It is an aim of an embodiment of the present invention to address the above problems and provide an immunoglobulin having affinity to integrins.

Definitions

The term “immunoglobulin” used herein may include a protein consisting of at least one polypeptide substantially encoded by an immunoglobulin gene. Recognised immunoglobulin genes include κ, λ, μ, γ, ζ, α, and ε constant genes and, in addition, the immunoglobulin variable region genes of which there are many. The immunoglobulin may exist as an antibody, both whole antibodies and biologically functional fragments thereof. Such biologically functional fragments retain at least one antigen binding region of a corresponding full-length antibody, i.e. with immuno-specificity for an integrin. The immunoglobulin may comprise a monoclonal antibody or functional fragment thereof.

Monovalent immunoglobulins are dimers (HL) comprising a heavy (H) chain associated by a disulphide bridge with a light chain (L). Divalent immunoglobulins are tetramers (H2L2) comprising two dimers associated by at least one disulphide bridge. Polyvalent immunoglobulins may also be produced, for example, by linking multiple dimers.

The basic structure of an immunoglobulin or antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen. On either side of the CDRs of the heavy and light chains is a framework region, a relatively conserved sequence of amino acids that anchors and orients the CDRs. The constant region consists of one of five heavy chain sequences (μ, γ, ζ, α, or ε) and one of two light chain sequences (κ or λ). The heavy chain constant region sequences determine the isotype of the antibody and the effector functions of the molecule.

As used herein, the terms “human immunoglobulin”, or “human monoclonal antibody” are intended to mean a monoclonal immunoglobulin or antibody comprising substantially the same heavy and light chain CDR amino acid sequences as found in a particular human immunoglobulin. An amino acid sequence which is substantially the same as a heavy or light chain CDR exhibits a considerable amount or extent of sequence identity when compared to a reference sequence. Such identity is definitively known or recognizable as representing the amino acid sequence of the particular human immunoglobulin or monoclonal antibody. Substantially the same heavy and light chain CDR amino acid sequence can have, for example, minor modifications or conservative substitutions of amino acids. Such a human immunoglobulin maintains its function of selectively binding an integrin antigen. The term “human monoclonal immunoglobulin” is intended to include a monoclonal immunoglobulin with substantially human CDR amino acid sequences produced, for example, by recombinant methods such as production be a phage library, by lymphocytes or by hybridoma cells. The term “recombinant human immunoglobulin” is intended to include a human immunoglobulin produced using recombinant DNA technology.

As used herein, the term “antigen binding region” is intended to mean a region of the immunoglobulin having specific binding affinity for an antigen. The binding region may be a hypervariable CDR or a functional portion thereof. By the term “functional portion” of a CDR, it is intended to mean a sequence within the CDR which shows specific affinity for an antigen. The functional portion of a CDR may comprise a ligand which specifically binds to an integrin, for example, the ‘RGD’ motif present in fibronectin, or the ‘AEIDGIEL’ present in Tenascin. Other integrin recognition ligands or motifs are described in Plow et. al. (J. Biol. Chem. Vol. 275 (29)), which is incorporated herein by reference.

As used herein, the term “CDR” is intended to mean a hypervariable region in the heavy and light variable chains. There may be one, two, three or more CDRs in each of the heavy and light chains of the immunoglobulins. Normally, there are at least three CDRs on each chain which, when configured together, form the antigen binding site, i.e. the three dimensional combining site with which the antigen binds or specifically reacts. It has been postulated that there may be four CDRs in the heavy chains of some antibodies.

The definition of CDR also includes overlapping or subsets of amino acid residues when compared against each other. The exact residue numbers which encompass a particular CDR or a functional portion thereof, will vary depending on the sequence and size of the CDR. Those skilled in the art can routinely determine which residues comprise a particular CDR given the variable region amino acid sequence of the antibody.

As used herein, the term “functional fragment”, when used in reference to a human immunoglobulin, is intended to refer to a portion of the immunoglobulin which retains a functional activity. A functional activity can be, for example, antigen binding activity or specificity. A functional activity can also be, for example, an effector function provided by an antibody constant region. Human monoclonal immunoglobulin functional fragments include, for example, individual heavy or light chains and fragments thereof, such as VL, VH and Fd; monovalent fragments, such as Fv, Fab, and Fab′; bivalent fragments such as F(ab′)₂; single chain Fv (scFv); and Fc fragments. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22: 189-224 (1993); Pluckthun and Skerra, Met-h. Enzymol., 178: 497-515 (1989) and in Day, E. D., Advanced Immunochemistry, Second Ed., Wiley-Liss, Inc., New York, N.Y. (1990), which are incorporated herein by reference.

The term “functional fragment” is intended to include, for example, fragments produced by protease digestion or reduction of a human monoclonal antibody and by recombinant DNA methods known to those skilled in the art.

The term “VL fragment” as used herein, refers to a fragment of the light chain of a human monoclonal antibody which includes all or part of the light chain variable region, including the CDRs. A VL fragment can further include light chain constant region sequences.

The term “VH fragment” as used herein, refers to a fragment of the heavy chain of a human monoclonal antibody which includes all or part of the heavy chain variable region, including the CDRs.

The term “Fd fragment” as used herein, refers to the light chain variable and constant regions coupled to the heavy chain variable and constant regions, i.e. VL CL and VH CH-1.

The term “Fv fragment”, as used herein, refers to a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” as used herein, refers to a monovalent antigen-binding fragment of a human monoclonal antibody that is larger than an Fv fragment. For example, an Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” as used herein, refers to a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” as used herein, refers to a bivalent antigen-binding fragment of a human monoclonal antibody. An F(ab′)₂ fragment includes, for example, all or part of the variable regions of two heavy chains and two light chains, and can further include all or part of the first constant domains of two heavy chains and two light chains.

One skilled in the art knows that the exact boundaries of a fragment of a human monoclonal antibody are not important, so long as the fragment maintains a functional activity. Using well-known recombinant methods, one skilled in the art can engineer a polynucleotide sequence to express a functional fragment with any endpoints desired for a particular application.

As used herein, the term “blood clotting disorder” is intended to include the disease state of thromboembolic disorder, i.e. an individual suffering from thromboembolism or excessive blood clotting (a thrombus), and the disease state of blood coagulation disorder, i.e. an individual suffering from too little blood clotting, for example, idiopathic thrombocytopenia.

As used herein, the term “inflammation” is intended to encompass any response reaction of the blood to an infection normally by the white blood cells in response to infection with a chemical.

As used herein, the term “label” is intended to mean a moiety that can be attached to a human immunoglobulin, or other molecule of the invention. Moieties can be used, for example, for therapeutic or diagnostic procedures. Therapeutic labels include, for example, moieties that can be attached to an immunoglobulin of the invention and used to monitor the binding of the immunoglobulin to an integrin. Diagnostic labels include, for example, moieties which can be detected by analytical methods.

Analytical methods include, for example, qualitative and quantitative procedures. Qualitative analytical methods include, for example, immunohistochemistry and indirect immunofluorescence. Quantitative analytical methods include, for example, immunoaffinity procedures such as radioimmunoassay, ELISA or FACS analysis. Analytical methods also include both in vitro and in vivo imaging procedures. Specific examples of diagnostic labels that can be detected by analytical means include enzymes, radioisotopes, fluorochromes, chemiluminescent markers, and biotin.

A label can be attached directly to an immunoglobulin of the invention, or be attached to a secondary binding agent that specifically binds a molecule of the invention. Such a secondary binding agent can be, for example, a secondary antibody. A secondary antibody can be either polyclonal or monoclonal, and of human, rodent or chimeric origin.

As used herein, the term “immunospecificity” means the binding region is capable of immunoreacting with an integrin, by specifically binding therewith. The immunoglobulin or functional fragment thereof can selectively interact with an antigen (integrin molecule) with an affinity constant of approximately 10⁻⁵ to 10⁻¹³ M⁻¹, preferably 10⁻⁶ to 10⁻¹⁰ M⁻¹, even more preferably, 10⁻⁷ to 10⁻⁹ M⁻¹. By the term “immunoreact”, we mean the binding region is capable of eliciting an immune response upon binding with an integrin, or epitope thereof.

As used herein, the term “epitope” means any region of an antigen with ability to elicit, and combine with, a binding region of the immunoglobulin.

As used herein, the term “effective amount” is intended to mean the amount of a molecule of the invention which can reduce a specific disease state, i.e. blood clotting disorder. The actual amount considered to be an effective amount for a particular application can depend, for example, on such factors as the affinity, avidity, stability, bioavailability or selectivity of the molecule, the moiety attached to the molecule, the pharmaceutical carrier and the route of administration. Effective amounts can be determined or extrapolated using methods known to those skilled in the art. Such methods include, for example, in vitro assays with cultured cells or tissue biopsies and credible animal models.

By the terms “substantially the amino acid/polynucleotide/peptide sequence”, we mean that the sequence has at least 60% sequence identity with the amino acid/polynucleotide/peptide sequences of any one of the sequences referred to. Calculation of percentage identities between different protein and DNA sequences may be carried out by the generation of multiple alignments by the Clustal program. An amino acid/polynucleotide/peptide sequence with a greater identity than 65% to any of the sequences referred to is also envisaged. An amino acid/polynucleotide/peptide sequence with a greater identity than 70% to any of the sequences referred to is also envisaged. An amino acid/polynucleotide/peptide sequence with a greater identity than 75% to any of the sequences referred to is also envisaged. An amino acid/polynucleotide/peptide sequence with a greater identity than 80% to any of the sequences referred to is also envisaged. Preferably, the amino acid/polynucleotide/peptide sequence has 85% identity with any of the sequences referred to, more preferably 90% identity, even more preferably 92% identity, even more preferably 95% identity, even more preferably 97% identity, even more preferably 98% identity and, most preferably, 99% identity with any of the referred to sequences.

A substantially similar nucleotide sequence will be encoded by a sequence which hybridizes to the sequences shown in SEQ ID No.s 1-10, 12 and 14-20, or their complements under stringent conditions. By stringent conditions, we mean the nucleotide hybridises to filter-bound DNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but preferably less than 100, 50, 20, 10, or 5 amino acids from the sequences shown in SEQ ID No.s 11 and 13.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change.

Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

The accurate alignment of protein or DNA sequences is a complex process which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantitate the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882;) are efficient ways to generate multiple alignments of proteins and DNA.

Frequently, automatically generated alignments require manual alignment, exploiting the trained user's knowledge of the protein family being studied, e.g., biological knowledge of key conserved sites. One such alignment editor program is Align (http://www.gwdg.de/˜dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. The percentage identity can be defined as: (N/T)*100 where N=the number of positions at which the two sequences share an identical residue, and T is the total number of positions compared. Residues in the alignment where one or both sequences have gaps are not included in the calculation. There are a number of programs that calculate this value for pairs within an alignment; For proteins, one is PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYLIP. Alternatively, percentage identity can be defined as (NIS)*100 where S is the length of the shorter sequence being compared.

Other modifications in protein sequences are also envisaged and within the scope of the claimed invention, i.e. those which occur during or after translation, e.g. by acetylation, amidation, carboxylation, phosphorylation, proteolytic cleavage or linkage to a ligand.

According to a first aspect of the present invention there is provided a recombinant human immunoglobulin or functional fragment thereof comprising at least one antigen binding region having an amino acid sequence independently selected from a group consisting of:—

(i) residues 31-35 of SEQ. ID No:11;

(ii) residues 50-66 of SEQ. ID No:11;

(iii) residues 100-110 of SEQ. ID No:11;

(iv) residues 31-33 of SEQ. ID No:13;

(v) residues 30-38 of SEQ. ID No:13;

(vi) residues 24-40 of SEQ. ID No:13;

(vii) residues 56-61 of SEQ. ID No:13; and

(viii) residues 95-102 of SEQ. ID No:13.

The antigen binding region may comprise a Complementarity Determining Region (CDR) of the immunoglobulin, or functional fragment thereof. Mutations may reside in framework regions between the CDRs of the immunoglobulin or functional fragment thereof.

According to a second aspect of the present invention, there is provided a recombinant human immunoglobulin or functional fragment thereof comprising at least one antigen binding region encoded by a polynucleotide having a nucleotide sequence independently selected from a group consisting of:—

(i) residues 91-105 of SEQ. ID No:10;

(ii) residues 148-198 of SEQ. ID No:10;

(iii) residues 298-330 of SEQ. ID No:10;

(iv) residues of 91-99 of SEQ. ID No:12;

(v) residues 88-114 of SEQ. ID No:12;

(vi) residues 70-120 of SEQ. ID No:12;

(vii) residues 166-183 of SEQ. ID No:12; and

(viii) residues 283-306 of SEQ. ID No:12.

According to a third aspect of the present invention there is provided a recombinant human immunoglobulin or functional fragment thereof comprising a light chain variable region (VL) and/or a heavy chain variable region (VH), the light chain variable region having an amino acid sequence which is substantially as set out in SEQ ID. No. 13, the heavy chain variable region having the amino acid sequence which is substantially as set out in SEQ ID. No. 11.

According to a fourth aspect of the present invention there is provided a recombinant human immunoglobulin or functional fragment thereof comprising a light chain variable region (VL) and/or a heavy chain variable region (VH), the light chain variable region being encoded by a polynucleotide having a nucleotide sequence which is substantially as set out in SEQ ID. No:12, the heavy chain variable region being encoded by a polynucleotide having a nucleotide sequence which is substantially as set out in SEQ ID. No:10.

According to a fifth aspect, there is provided an isolated peptide comprising an amino acid sequence independently selected from a group consisting of:—

(i) residues 31-35 of SEQ. ID No:11;

(ii) residues 50-66 of SEQ. ID No:11;

(iii) residues 100-110 of SEQ. ID No:11;

(iv) residues 31-33 of SEQ. ID No:13;

(v) residues 30-38 of SEQ. ID No:13;

(vi) residues 24-40 of SEQ. ID No:13;

(vii) residues 56-61 of SEQ. ID No:13; and

(viii) residues 95-102 of SEQ. ID No:13.

The peptide may comprise a polypeptide. Preferably, the peptide is adapted to bind to an integrin.

According to a sixth aspect of the present invention, there is provided an isolated polynucleotide comprising a nucleotide sequence independently selected from a group consisting of:—

(i) residues 91-105 of SEQ. ID No:10;

(ii) residues 148-198 of SEQ. ID No:10;

(iii) residues 298-330 of SEQ. ID No:10;

(iv) residues of 91-99 of SEQ. ID No:12;

(v) residues 88-114 of SEQ. ID No:10;

(vi) residues 70-120 of SEQ. ID No:12;

(vii) residues 166-183 of SEQ. ID No:12; and

(viii) residues 283-306 of SEQ. ID No:12.

Preferably, the polynucleotide encodes a recombinant human immunoglobulin, or functional fragment thereof. Preferably, the polynucleotide comprises a nucleotide sequence substantially encoding an amino acid sequence of at least one antigen binding region of the recombinant human immunoglobulin, or functional fragment thereof.

Advantageously, the immunoglobulin or functional fragment thereof has a potential therapeutic interaction in its own right, and is an improvement on current therapies which use immunoglobulins comprising a non-human region, for example, murine, Fc fragment (framework regions), or at least one murine antigen binding region or Complimentarity Determining Region (CDR).

According to a seventh aspect, there is provided a recombinant immunoglobulin or functional fragment thereof defined in any of the first to fourth aspects, a peptide defined in the fifth aspect, or a polynucleotide defined in the sixth aspect, each being optionally derivatised, for use as a medicament or in diagnosis.

The immunoglobulin or functional fragment thereof defined in any of the first to fourth aspects, the peptide defined in the fifth aspect, or the polynucleotide defined in the sixth aspect, may not be derivatised.

Preferably, the medicament is adapted to retard or prevent a blood clotting disorder.

According to an eighth aspect, there is provided use of a recombinant immunoglobulin or functional fragment thereof defined in any of the first to fourth aspects, a peptide defined in the fifth aspect, or a polynucleotide defined in the sixth aspect, optionally after derivatisation, for the preparation of a medicament for the treatment of a blood clotting disorder.

Hence, the immunoglobulin or functional fragment thereof, peptide or polynucleotide may be modified prior to use, preferably to produce a derivative or variant thereof. The immunoglobulin or functional fragment thereof defined in any of the first to fourth aspects, the peptide defined in the fifth aspect, or the polynucleotide defined in the sixth aspect, may not be derivatised.

Preferably, the medicament is adapted to retard or prevent thromboembolic disorder, and/or inflammation.

According to a ninth aspect, there is provided a method of treating blood clotting disorders and/or inflammation, the method comprising administering to a patient a recombinant immunoglobulin or functional fragment thereof defined in any of the first to fourth aspects, a peptide defined in the fifth aspect, or a polynucleotide defined in the sixth aspect, each being optionally derivatised.

The method of treating may comprise anti-inflammation and/or anti-thromboembolic therapy.

According to a tenth aspect, there is provided a pharmaceutical composition comprising a recombinant immunoglobulin or functional fragment as defined in any of the first to fourth aspects of the invention, a peptide defined in the fifth aspect, or a polynucleotide defined in the sixth aspect, each being optionally derivatised, and a pharmaceutically acceptable excipient, carrier, buffer or stabiliser.

The immunoglobulin or functional fragment thereof defined in any of the first to fourth aspects, the peptide defined in the fifth aspect, or the polynucleotide defined in the sixth aspect, may not be derivatised.

Suitable pharmaceutical excipients include, for example, aqueous solutions such as physiologically buffered saline, and other solvents or media such as glycols, glycerol, oils or injectable organic esters. A pharmaceutical carrier can contain a physiologically acceptable compound that acts, for example, to stabilize or increase the solubility of a pharmaceutical composition. Such a physiologically acceptable compound can be, for example, a carbohydrate, such as glucose, sucrose or dextrose; an antioxidant, such as ascorbic acid or glutathione; a chelating agent; a low molecular weight protein; or another stabilizer or excipient. Those skilled in the art will know that the choice of the pharmaceutical medium and the appropriate preparation of the composition will depend on the intended use and mode of administration.

Preferably, the composition is adapted to be administered to a patient in order to prevent or reduce a blood clotting disorder and/or inflammation in the said patient. Preferably, the blood clotting disorder comprises thromboembolic disorder in the patient. Preferably, the composition can also be used to detect blood clotting disorders, more preferably, thromboembolic disorders and/or inflammation in the patient.

According to an eleventh aspect, there is provided a kit for treating or diagnosing an individual having a blood clotting disorder, the said kit comprising a recombinant immunoglobulin or functional fragment thereof defined by any of the first to fourth aspects, a peptide defined in the fifth aspect, or a polynucleotide defined in the sixth aspect.

Preferably, the kit further comprises detection means which preferably, comprises an assay adapted to detect the presence and/or absence of an antigen specific to the immunoglobulin or functional fragment thereof, peptide or polynucleotide. The kit may comprise a label which may be detected by the detection means.

According to a twelfth aspect, there is provided a method for determining an individual's susceptibility to a blood clotting disorder and/or inflammation, the method comprising:—
(i) obtaining a sample from an individual, and
(ii) detecting the level of antigen in the sample using a recombinant immunoglobulin or fragment thereof defined by any of the first to fourth aspects, a peptide defined in the fifth aspect, or a polynucleotide defined in the sixth aspect.

Preferably, the antigen comprises an integrin, more preferably, GPIIb/IIIa. The sample may comprise blood, urine, tissue etc.

The recombinant immunoglobulin or functional fragment thereof may comprise at least two, suitably at least three, more suitably at least four antigen binding regions defined in the first and second aspects. Preferably, the recombinant immunoglobulin or functional fragment comprises at least five, more preferably at least six, and even more preferably at least seven antigen binding regions. In a preferred embodiment, the recombinant immunoglobulin or functional fragment comprises all eight of the antigen binding regions.

The recombinant immunoglobulin may comprise any, or all, of the antigen binding regions. The recombinant immunoglobulin may comprise an antigen binding site with which the antigen binds, preferably eliciting an immunological response. Preferably, the at least one antigen binding region forms at least part of the antigen binding site.

Preferably, the immunoglobulin or functional fragment thereof is monovalent. However, the immunoglobulin may be divalent or polyvalent.

Disadvantageously, divalent or polyvalent immunoglobulins may have a tendency to act as a fibrinogen substitute and promote the aggregation of platelets. This may happen because a divalent immunoglobulin is able to bind to a first fibrinogen receptor on a first platelet cell and also a second fibrinogen receptor on a second platelet cell. Hence, a bridge is formed between the two platelet cells by the divalent immunoglobulin, thereby resulting in platelet aggregation. This may be dangerous.

Advantageously, and in contrast to divalent and polyvalent immunoglobulins discussed above, monovalent immunoglobulins are only able to bind to one fibrinogen receptor on a single platelet thereby preventing fibrinogen binding thereto and, hence, prevents platelet aggregation. Monovalent immunoglobulin are unable to form bridges between platelet cells.

Thus, the invention may extend to a method of making a recombinant immunoglobulin or functional fragment thereof defined in any of the first to fourth aspects, wherein the immunoglobulin or functional fragment thereof is monovalent.

Preferably, the immunoglobulin or functional fragment thereof is made directly as a monovalent immunoglobulin or functional fragment thereof. Preferably, the method does not involve cleaving a divalent or polyvalent immunoglobulin to produce the monovalent immunoglobulin. Such an approach would normally result in a monovalent immunoglobulin having a lower affinity for the ligand than a monovalent immunoglobulin which has been made directly.

Preferably, a monovalent immunoglobulin has a relatively short life-span in vivo in the human body, for example, preferably, less than 2 months, more preferably, less than 1 month, even more preferably, less than 2 weeks and, most preferably less than 48 hours. The immunoglobulin may be used medically by administering to a patient post-operation, for example, to decrease the risk of the patient suffering from thrombosis after an operation. Divalent and polyvalent immunoglobulins tend to have much longer life-spans, for example, approximately 2-3 months which are less suitable for medical use. In addition, divalent immunoglobulin tend to cause thrombosis in patients.

Preferably, the antigen comprises an integrin.

Integrins comprise an α and β chain, preferably drawn from two families. Recombination of these chains results in a variety of integrin molecules with differing functions in the immune and coagulatory responses.

The α chains include: α₁, α₂, α₃, α₄, α₅, α₆, α₇, α₈, α₉, α₁₁, α_{Ib}, α_{IIb}, α_{M}, α_{V}, α_{E}, α_{L}, α_{X}.

The β chains include: β₁, β₂, β₃, β₄, β₅, β₆, β₇, β₈.

The α and β chains may be combined to produce a specific combination, each resulting in an integrin, for example:—

α_{V}β₃, α_{V}β₅, α_{IIb}β₃, α_{M}β₂, α₁β₁, α₂β₁, α_{Ib}β₃, α₅β₁, α₈β₁, α₉β₁, α_{V}β₆, α_{E}β₇, α₃β₁, α₄β₁, α₄β₇, α₅β₁, α₈β₁, α_{V}β₁, α_{V}β₆, α_{X}β₂, α_{L}β₂, α₆β₁, α₇β₁, α₆β₄.

Hence, there are approximately twenty four integrin family members.

Preferably, the antigen comprises an integrin independently selected from a group consisting of α_{V}β₃, α_{V}β₅, α_{IIb}β₃, α_{M}β₂, α₁β₁, α₂β₁, α_{Ib}β₃, α₅β₁, α₈β₁, α₉β₁, α_{V}β₆, α_{E}β₇, α₃β₁, α₄β₁, α₄β₇, α₅β₁, α₈β₁, α_{V}β₁, α_{V}β₈, α_{X}β₂, α_{L}β₂, α₆β₁, α₇β₁, and α₆β₄.

Preferably, the antigen comprises a β3 integrin. Preferably, the antigen comprises an integrin independently selected from a group consisting of α_{V}β₃, α_{IIb}β₃, and α_{Ib}β₃.

For example, suitable integrins may include glycoprotein IIb/IIIa (α_{IIb}β₃), LFA-1 (α_{L}β₂), VLA-4 (α₄β₁), MAC-1 (α_{M}β₂) and p150.95 (α_{X}β₂). The aforementioned integrins are structurally, functionally and immunochemically similar with each other. Therefore, the other integrins listed above which may be structurally, functionally and immunochemically similar to those mentioned above, are also within the scope of the claimed invention.

Preferably, and advantageously, the immunoglobulin or functional fragment thereof has immunospecificity for an integrin, for example, human glycoprotein (GP) IIb/IIIa, preferably, when the said integrin is substantially purified. Preferably, and advantageously, the immunoglobulin or functional fragment thereof, is adapted to bind to a platelet having an integrin, for example, human glycoprotein IIb/IIIa, on an outer surface thereof.

The recombinant immunoglobulin or functional fragment thereof may be isolated, preferably from an individual suffering from autoimmune idiopathic thrombocytopenia (AITP). This disease state (AITP) is characterised by the patient having a low platelet concentration relative to that in a healthy individual. Furthermore, the disease state AITP is characterised by the presence of immunoglobulins with immunospecificity against platelet integrins such as Glycoprotein (GP) IIb/IIIa. Immunoglobulins with immunospecificity against other platelet integrins may also be present in an AITP sufferer.

Preferably, and advantageously, the immunoglobulin or functional fragment thereof is adapted to substantially inhibit aggregation of human platelets in response to an agonist. Preferably, and advantageously, the immunoglobulin or functional fragment thereof is adapted to substantially inhibit the binding of fibrinogen to glycoprotein IIb/IIIa, or epitopes thereof, the major interaction in initiating platelet aggregation.

Interestingly, the immunoglobulin or functional fragment thereof is derived by somatic mutation. Preferably, the immunoglobulin or functional fragment thereof is derived by affinity maturation, as opposed to germline evolution.

An individual with AITP normally has a low platelet count caused by accelerated clearance of platelets, although antibodies that inhibit function have been reported. It is however surprising that an antibody appears to arise via somatic mutation to contain motifs that specifically inhibits the interaction between GPIIb/IIIa and fibrinogen. Whilst the isolated monomeric form inhibits platelet aggregation, the dimeric form that normally exists in vivo might be expected to accelerate platelet aggregation thus blocking the normal pathology of the disease.

The nomenclature used for the CDRs of the present invention are shown in Table:1.

TABLE 1
The Complementarity Determining Regions
(hypervariable regions) of the immunoglobulin

| Nomen- | | SEQ. | | SEQ. | Base |
|---|---|---|---|---|---|
| clature | Description | ID No | Residues | ID No | Number |
| VH-CDR1 | Heavy chain - | 11 | 31-35 | 10 | 91-105 |
| | variable | | | | |
| VH-CDR2 | Heavy chain - | 11 | 50-66 | 10 | 148-198 |
| | variable | | | | |
| VH-CDR3 | Heavy chain - | 11 | 100-110 | 10 | 298-330 |
| | variable | | | | |
| VL-CDR1 | Light chain - | 13 | 24-40 | 12 | 70-120 |
| | variable | | | | |
| VL-CDR2 | Light chain - | 13 | 56-61 | 12 | 166-183 |
| | variable | | | | |
| VL-CDR3 | Light chain - | 13 | 95-102 | 12 | 283-306 |
| | variable | | | | |

Preferably, the antigen binding region defined by residues 31-33 of SEQ ID No.13 comprises an amino acid sequence ‘RSD’. Preferably, the binding region defined by the amino acid sequence as set out as residues 30-38 of SEQ ID No.13 comprises an amino acid sequence of ‘ARSDGVSLM’.

Preferably, functional fragments of the immunoglobulin comprise fragments with substantially the same heavy and light chain variable regions as the human immunoglobulin. Preferably, the functional fragment is integrin-specific. Preferably, the functional fragment includes fragments wherein at least one of the binding region sequences is substantially the same amino acid sequence as the binding region sequences of the immunoglobulin, more preferably, the integrin-specific human immunoglobulin. The functional fragment may comprise any of the fragments independently selected from a group consisting of VH, VL, Fd, Fv, Fab, Fab′, F (ab′)₂ and Fc fragment.

The functional fragment may comprise any one of the antigen binding region sequences of the VL, any one of the antigen binding region sequences of the VH, or a combination of VL and VH antigen binding regions of a human immunoglobulin. The appropriate number and combination of VH and VL antigen binding region sequences may be determined by those skilled in the art depending on the desired affinity and specificity and the intended use of the functional fragment.

Functional fragments of immunoglobulins may be readily produced and isolated using methods well known to those skilled in the art. Such methods include, for example, proteolytic methods, recombinant methods and chemical synthesis.

Proteolytic methods for the isolation of functional fragments comprise using human immunoglobulins as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F(ab′) fragment. An F(ab′)₂ fragment of the invention may be further reduced using, for example, DTT or 2-mercaptoethanol to produce two monovalent Fab′ fragments.

Functional fragments produced by proteolysis may be purified by affinity and column chromatographic procedures. For example, undigested antibodies and Fc fragments may be removed by binding to protein A. Additionally, functional fragments may be purified by virtue of their charge and size, using, for example, ion-exchange and gel filtration chromatography. Such methods are well known to those skilled in the art.

The human immunoglobulin or functional fragment thereof may be produced by recombinant methodology. Preferably, one initially isolates a polynucleotide encoding desired regions of the immunoglobulin heavy and light chains. Such regions may include, for example, all or part of the variable region of the heavy and light chains. Preferably, such regions can particularly include the antigen binding regions of the heavy and light chains, preferably the antigen binding sites, most preferably, the CDRs.

The polynucleotide encoding the human immunoglobulin or functional fragment of the invention may be produced using methods known to those skilled in the art. The polynucleotide encoding the immunoglobulin or a functional fragment thereof may be directly synthesized by methods of oligonucleotide synthesis known in the art. Alternatively, smaller fragments may be synthesized and joined to form a larger functional fragment using recombinant methods known in the art.

Preferably, the immunoglobulin or functional fragment thereof is produced by a bacteriophage expression system. Preferably, the bacteriophage expression system comprises a phage display library.

A useful procedure for isolating the polynucleotide which encodes the immunoglobulin or functional fragment thereof begins with isolation of cDNA which can be reverse-transcribed from RNA isolated from an individual suffering from autoimmune idiopathic thrombocytopenia (AITP). This disease state (AITP) is characterised by the presence of antibodies with immunospecificity against platelet integrins including Glycoprotein (GP) IIb/IIIa. Methods for cDNA synthesis are well known in the art. A cDNA encoding an immunoglobulin or functional fragment thereof including a heavy or light chain can be amplified using, for example, the polymerase chain reaction (PCR), preferably reverse transcription PCR (RT-PCR).

Suitable primers for PCR may be determined by those skilled in the art using conserved sequences which flank the particular functional fragment of a heavy or light chain. For example, suitable PCR primers may comprise any of the polynucleotides substantially as set out as SEQ ID No:1 to SEQ ID No:7, and SEQ ID No:14 to SEQ ID No:20. Suitable PCR conditions may be determined by those skilled in the art.

Preferably, the PCR is adapted to amplify the heavy chain, more preferably the VH CH-1 fragment, and even more preferably, the heavy chain variable fragment (VH). Alternatively, or additionally, the PCR is adapted to amplify the light chain, more preferably the VL CL fragment, and even more preferably, the light chain variable fragment (VL).

Preferably, the PCR reaction comprised using suitable primers, for example, which may be independently selected from a group of primers consisting of SEQ ID No: 1-7, and 14-20.

Preferably, the PCR products are cloned into a suitable expression vector, more preferably a phage expression vector, for example, pComb3HSS. Preferably, the heavy fragment is digested with xhoI and, preferably SpeI, prior to cloning into the vector. Preferably, the light fragment is digested with SacI and preferably, XbaI prior to cloning into the expression vector.

Preferably, the vector is introduced into a suitable host, for example, E. coli, for expression of the heavy and preferably, the light fragment, to occur. A suitable vector and host cell system can allow, for example, co-expression and assembly of functional fragments of the heavy and light chains. Preferably, the vector is introduced into the host by electroporation.

Other suitable systems for the expression of antibody fragments can be determined by those skilled in the art and include, for example, M13 phage expression vectors. Recombinant immunoglobulins' or functional fragments thereof can be substantially purified using methods known in the art, and which depend on the particular vector and host expression system used.

In a preferred embodiment, the invention is directed to an integrin specific human immunoglobulin. Advantageously, the immunoglobulin is human in origin, and is therefore likely to minimise any immune response upon administration to a human patient in contrast to using immunoglobulin comprising non-human elements. In addition, the immunoglobulin or functional fragment thereof, and polynucleotide and amino acid sequences encoding the immunoglobulin or functional fragment thereof, may be effectively used for the manufacture of compositions and diagnostics, and uses thereof, for example, in response to anti-inflammation and blood clotting disorder disease states.

According to a fourteenth aspect of the present invention, there is provided a recombinant DNA molecule comprising a polynucleotide defined in the sixth aspect, or derivative thereof.

Preferably, the recombinant DNA molecule comprises an expression vector. Preferably, the polynucleotide sequence is operatively linked to an expression control sequence. A suitable control sequence may comprise a promotor, an enhancer etc.

According to an fifteenth aspect of the present invention, there is provided a cell containing a recombinant DNA molecule of the fourteenth aspect.

The cell may be transformed or transfected with the recombinant DNA molecule by suitable means.

According to a sixteenth aspect of the present invention, there is provided a method of preparing a recombinant immunoglobulin or functional fragment thereof, the method comprising—
(i) culturing at least one cell defined in the fifteenth aspect capable of expressing the required immunoglobulin or functional fragment thereof; and
(ii) isolating the immunoglobulin or functional fragment thereof.

The immunoglobulin or functional fragment thereof may be used to act as a framework for the development of immunoglobulins showing immunospecificity against other members of the integrin family. Integrins comprise an a and β chain drawn from two families. Recombination of these chains results in a variety of integrin molecules with differing functions in the immune and coagulatory responses. Examples of integrins include:—

α_{V}β₃, α_{V}β₅, α_{IIb}β₃, α_{M}β₂, α₁β₁, α₂β₁, α_{Ib}β₃, α₅β₁, α₈β₁, α₉β₁, β_{V}β₆, α_{E}β₇, α₃β₁, α₄β₁, α₄β₇, α₅β₁, α₈β₁, α_{V}β₈, α_{X}β₂, α_{L}β₂, α₆β₁, α₇β₁, α₆β₄.

Each of these combinations has a variety of functions apart from their role in platelet aggregation, including activation of an immune response, leukocyte migration (both of these are anti-inflammatory therapeutic targets), the interaction between sperm and egg and angiogenesis (a prime anti-tumour target).

According to a seventeenth aspect, there is provided a method of isolating a recombinant immunoglobulin or functional fragment thereof having ability to bind to a target integrin, the method comprising:—
(i) mutating a recombinant immunoglobulin or functional fragment thereof as defined in any of the first to fourth aspects to produce a mutant, and
(ii) selecting the mutant for activity against a target integrin.

In a first embodiment, said mutating may comprise random mutagenesis, preferably using degenerative PCR. Preferably, cDNA for the immunoglobulin is used as a template in a PCR reaction which may be doped with a mutagen. Preferably, the PCR reaction is doped with a mutagenic nucleoside triphosphate, for example, dP and 8-oxo-2′deoxyguanosine. Advantageously, this allows the introduction of mutations in a highly controlled manner throughout the cDNA to produce a mutant library. The resultant library of mutants may be displayed on the surface of a phage, and antibodies may be selected against a desired integrin.

The resultant library of mutant antibodies may be selected against a desired integrin using biopanning. An ELISA plate may be coated with the desired integrin. For example, 100 μl of a 1 μgml⁻¹ solution of the desired integrin in bicarbonate buffer pH 8.6, and incubated overnight at 4° C.

Preferably, after washing twice with TBS, the plate may be blocked with 5% BSA in PBS and incubated for one hour at 37° C. After two further washes, 100 μl phage suspension may be added to each well and the plate incubated for two hours at 37° C.

The phage may be removed and the wells filled with TBS 0.05% Tween 20 (TBST) and pipetted vigorously. After 5 minutes the TBST may be removed, and for a first round of panning, the plate may be washed by this method once. In a second round of panning, 5 washes may be used, and in a third and subsequent rounds 10 washes were used. The phage may then be eluted with 50 μl of elution buffer per well and incubated at room temperature for 10 minutes. After vigorously pipetting, eluted phage may be removed and neutralised with 3 μl of 2M Tris base.

In a second embodiment, said mutating may comprise introducing at least one ligand having immunospecificity against the target integrin into at least one antigen binding region of the recombinant immunoglobulin or functional fragment thereof.

The at least one ligand may be independently selected from a group of ligands consisting of RDG, RSG, RSD, HHLGGAKQAGDV, GPR, RPG, AEIDGIEL, ARSDGVSLM, QIDS, LDT, IDAPS, DLXXL, and GFOGER. GFOGER is hydroxyproline.

The at least one antigen binding region may be in the heavy and/or light chain variable fragment. Preferably, the at least one ligand is introduced into any of the antigen binding regions in the heavy chain of the immunoglobulin or functional fragment thereof. Preferably, the at least on ligand is introduced into the first binding region. For example, see Table 1.

The ligand may be inserted by restriction enzyme digestion at an appropriate site determined by a variety of techniques including molecular modelling. A polynucleotide sequence encoding the ligand peptide sequence may be ligated into the cut restriction site. The exact details of this depends on the nature of ligand and the CDR being used.

In the second embodiment, said mutating may further comprise random mutagenesis.

According to an eighteenth aspect, there is provided a library or panel of recombinant immunoglobulins or functional fragments thereof, generated using a method defined in the seventeenth aspect.

A number of the integrin combinations listed above can be over-expressed in a variety of diseases particularly in tumours and, therefore immunoglobulins or antibodies can be used to localise the tumours. It is also suggested that the outcome of tumours and possibly other diseases may be predicted by the expression of integrins.

According to a nineteenth aspect, there is provided a method of isolating an anti-platelet immunoglobulin or functional fragment thereof comprising:—
(i) contacting at least one immunoglobulin or functional fragment thereof against at least one whole platelet; and
(ii) isolating an immunoglobulin or functional fragment thereof having binding specificity for the whole platelet.

By the term “whole platelet”, we mean complete or intact platelet which is preferably in situ with a platelet membrane, as opposed to regions or portions of a platelet which may not be in situ with the platelet membrane. By the term anti-platelet immunoglobulin, we mean an immunoglobulin or functional fragment thereof which is adapted to substantially inhibit aggregation of human platelets in response to an agonist. Preferably, and advantageously, the anti-platelet immunoglobulin or functional fragment thereof is adapted to substantially inhibit the binding of fibrinogen to glycoprotein IIb/IIIa, or epitopes thereof, the major interaction in initiating platelet aggregation.

Preferably, the at least one immunoglobulin is associated with a bacteriophage. Preferably, a bacteriophage library is contacted against the at least one whole platelet. Preferably, a plurality of whole platelets are used. Preferably, the said contacting comprises panning the phage library against the whole platelets.

Advantageously, the method is much less time-consuming than screening the immunoglobulins against a specific antigen such as GPIIb/IIIa in the search for functionally active immunoglobulins, because the integrin will be in situ with the platelet membrane.

All of the features described herein may be combined with any of the above aspects, in any combination.

An embodiment of the present invention will now be described, by way of example, with reference to the accompanying drawings in which:—

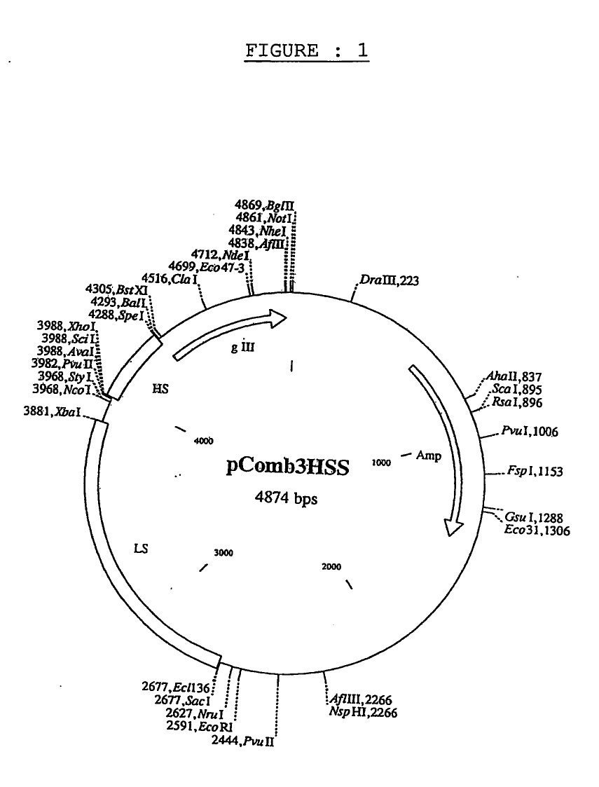

FIG. 1 is a schematic representation of pComB3HSS phage display vector;

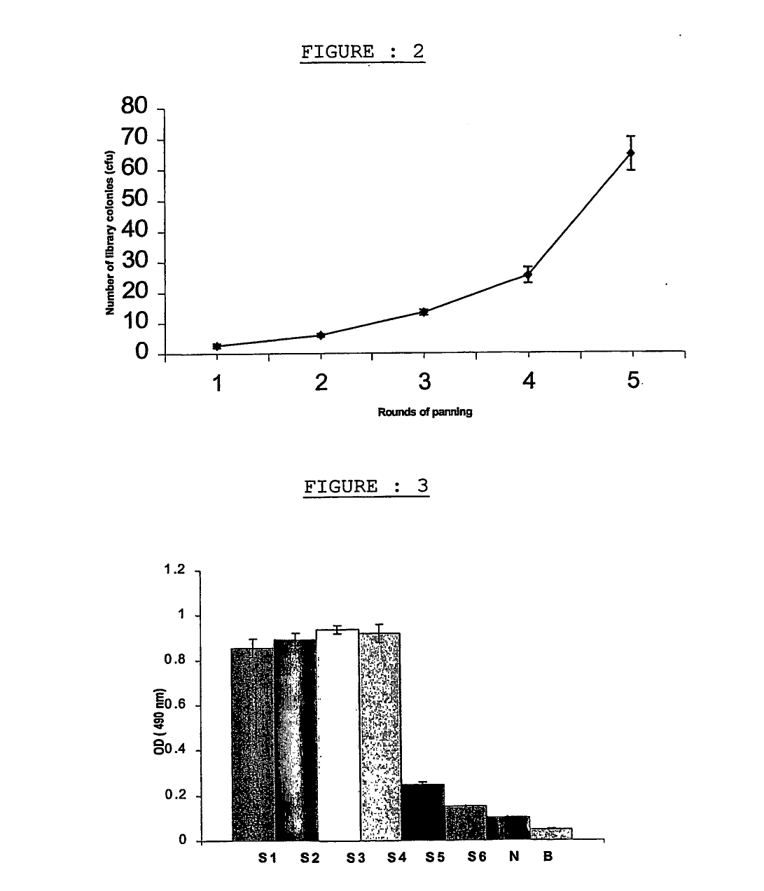

FIG. 2 illustrates enrichment of phage during five consecutive rounds of biopanning;

FIG. 3 illustrates the reactivity of phage against whole platelets;

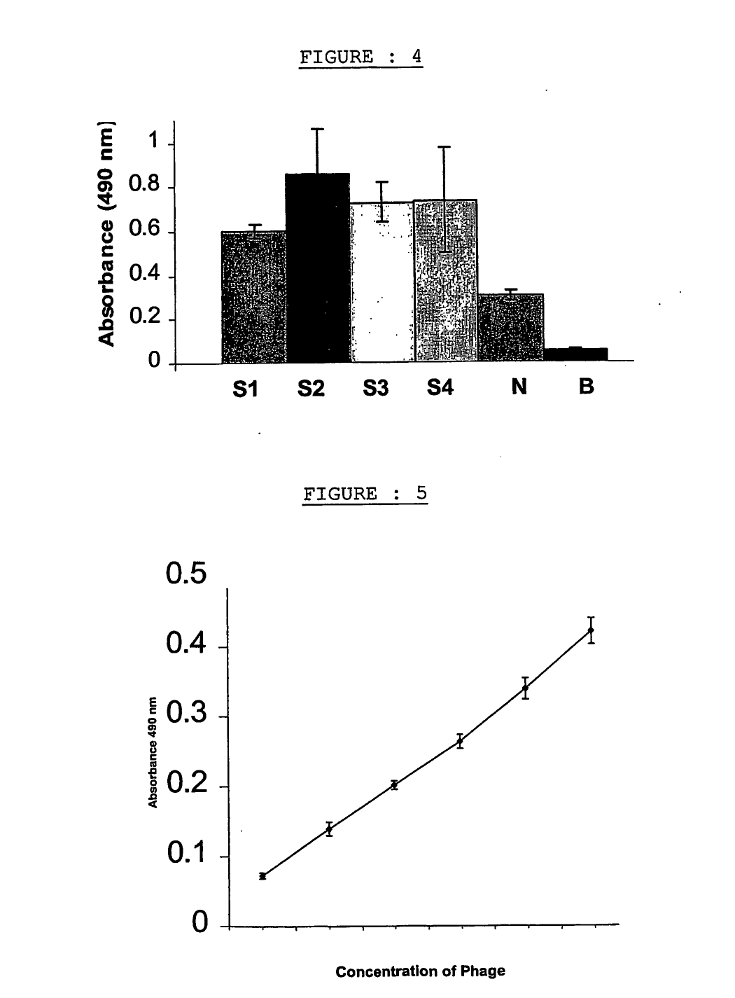

FIG. 4 illustrates the reactivity of reactive phage against platelet lysate preparation;

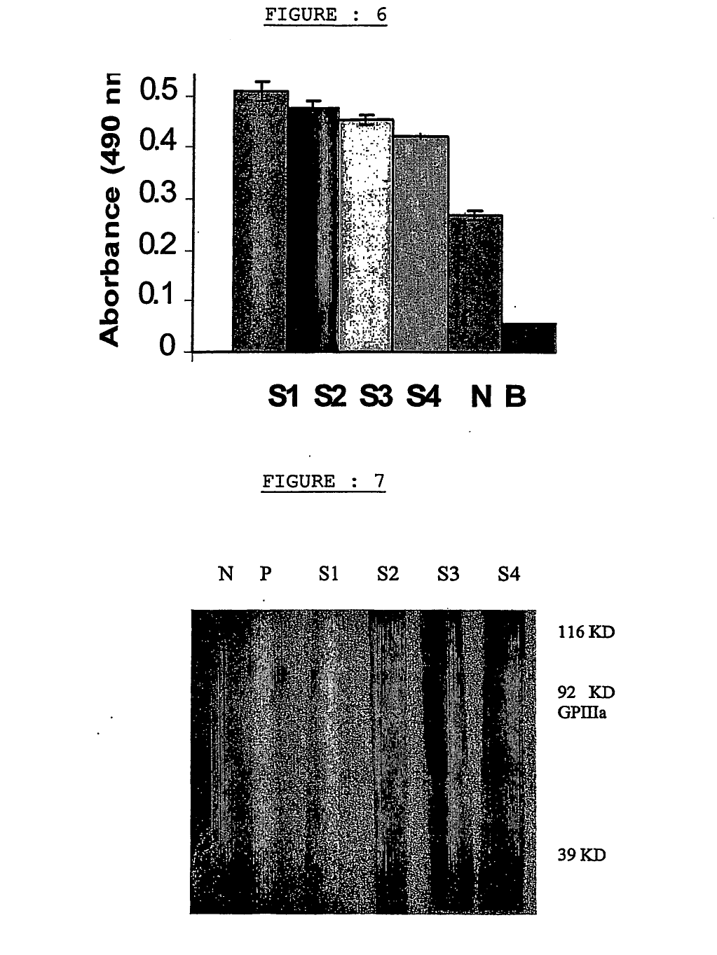

FIG. 5 illustrates a titration of Fab bearing phage against platelet lysate preparation;

FIG. 6 illustrates the reactivity of phage against platelet glycoprotein (GP) IIb/IIIa;

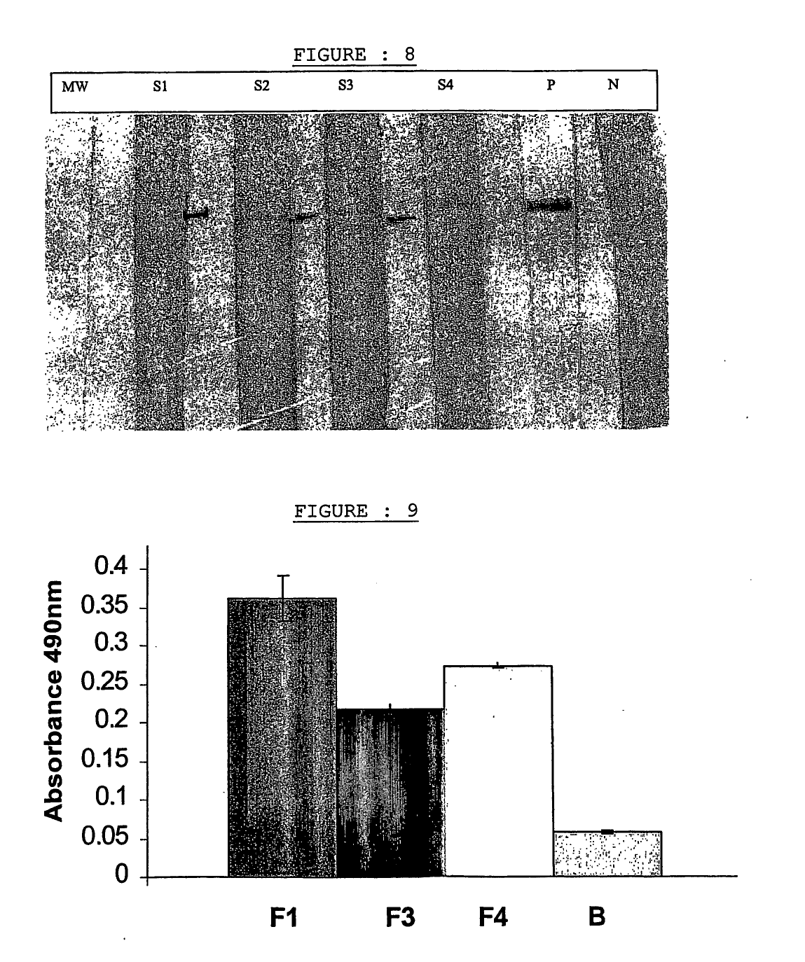

FIG. 7 illustrates a Western blot of non-reduced human platelet membrane lysate reacting against four anti-platelet Fab clones with positive (P) and negative (N) controls;

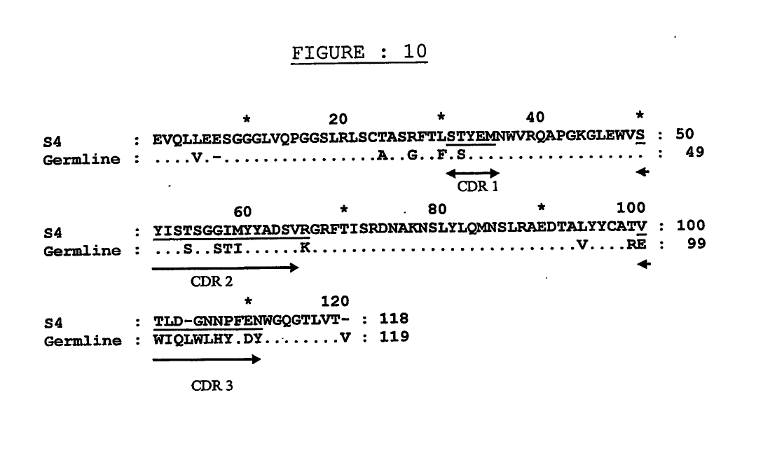

FIG. 8 illustrates a Western blot of non-reduced human platelet glycoprotein IIb/IIIa against four anti-platelet Fab clones with positive (P) and negative (N) controls;

FIG. 9 illustrates an ELISA assay to detect the reactivity of isolated soluble Fab anti-platelet antibodies against intact washed platelets;

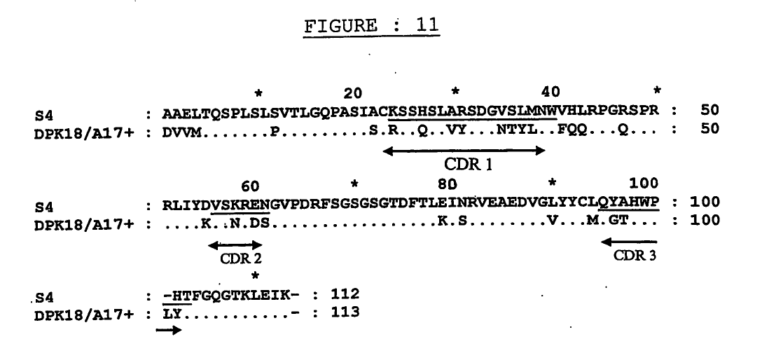

FIG. 10 illustrates alignment of amino acid sequence of Heavy chain of expressed antibody with germline gene amino acid sequence;

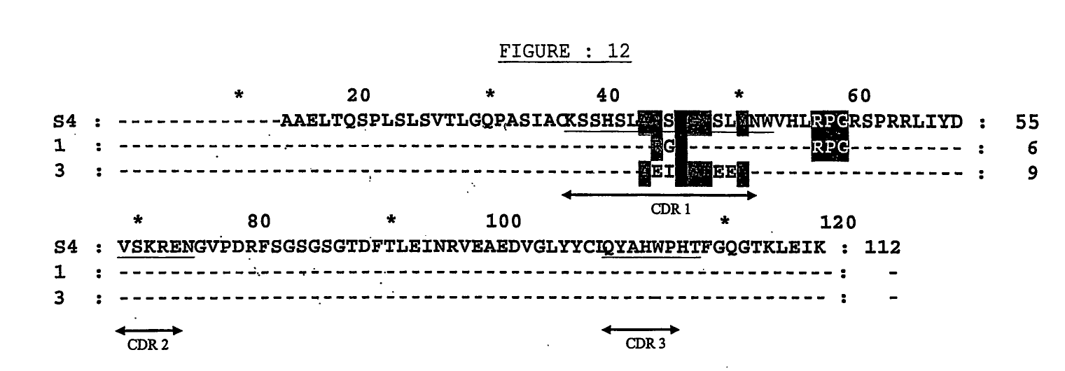

FIG. 11 illustrates alignment of amino acid sequence of Light chain of expressed antibody with germline gene amino acid sequence;

FIG. 12 illustrates alignment of amino acid sequence of Light chain of expressed antibody with putative ligand mimitope amino acid sequences;

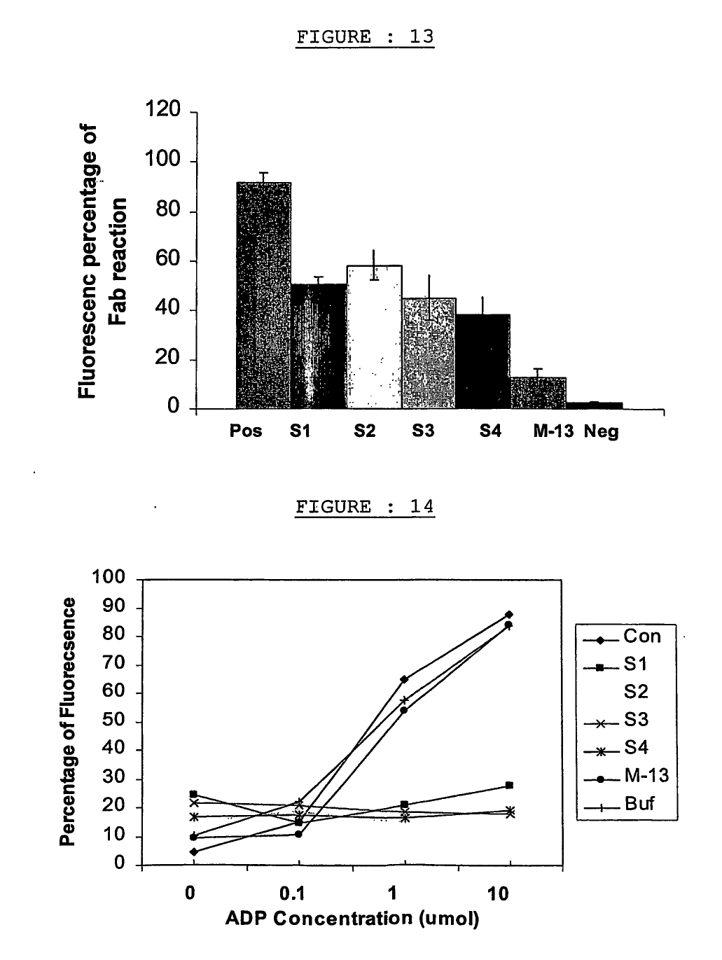

FIG. 13 illustrates flow cytometric analysis of platelet binding activity of four clones isolated from Fab library;

FIG. 14 illustrates flow cytometric analysis to determine the ability of platelet reactive Fab bearing phage to inhibit the binding of fibrinogen to resting and activated platelet; and

to the Sequence Listing.

An embodiment of the present invention will now be described, by way of the following example.

### EXAMPLE

The first step of the project was to construct combinatorial antibody libraries from two patients with autoimmune idiopathic thrombocytopenia (AITP). This disease state is (AITP) is characterised by the patient having a low platelet concentration relative to a healthy individual. Furthermore, the disease state AITP is characterised by the presence of antibodies with immunospecificity against the platelet integrin Glycoprotein (GP) IIb/IIIa.

Library Construction

Total RNA was isolated from homogenised splenic tissue from the AITP patient using an Ultraspec™ RNA isolation kit (Biotex Laboratories, UK). cDNA was then produced by carrying out reverse transcription on the RNA isolated from the splenic tissue as follows.

10-30 μg of isolated RNA was added to a sterile 1.5 ml Eppendorf tube. 1 μg (2 μl) oligodT were then added, and the volume made up to 27 μl with nuclease free water, or DEPC (diethyl pyrocarbonate) water. The reactants were heated at 70° C. for 10 min and then cooled to 40° C.

2 μl of RNAse inhibitor was then added with 10 μl (5×) RT buffer. 3 μl dNTP's were then added (2 mM each of dATP, dCTP, dGTP, dTTP). 5 μl of 0.1M DTT was then added and the volume made up to 48 μl using DEPC water, before adding reverse transcriptase enzyme. 200 Units (2 μl) reverse transcriptase (SuperscriptII™, <Gibco, UK) was then added and the reactants incubated at room temperature for 10 minutes. The reaction was terminated by incubating at 90° C. for 5 minutes, and then at 4° C. for 10 minutes.

Following reverse transcription of the AITP patients' RNA, the variable heavy region (VH) and variable light (x) chains of the cDNA were amplified by PCR using constant (C) region primers and a panel of variable (VH and Vκ) first framework specific primers, and additional VH and VK primers.

The heavy chain variable region primers which start the amplification at the COOH terminus are:—

| | |
|---|---|
| VH1f | |
| 5′-caggtgcagctgctcgagtctggg-3′; | (SEQ ID No 14) |
| VH2f: | |
| 5′-caggtgcagctactcgagtcggg-3′; | (SEQ ID No 1) |
| VH3a | |
| 5′-gaggtgcagctcgaggagtctggg-3′; | (SEQ ID No 15) |
| VH4f: | |
| 5′-caggtgcagctgctcgagtcggg-3′; | (SEQ ID No 2) |
| VH4g | |
| 5′-caggtgcagctactcgagtgggg-3′; | (SEQ ID No 16) |
| VH6a: | |
| 5′-caggtacagctcgagcagtcagg-3′; | (SEQ ID No 3) |
| and | |
| VH6f: | |
| 5′-caggtacagctgctcgagtcaggtcca-3′. | (SEQ ID No 4) |

The above heavy chain variable region primers were used in a PCR reaction with a heavy chain IgG1-specific constant (CH₁) domain primer:—

| | |
|---|---|
| CG1z | |
| 5′-gcatgtactagttttgtcacaagatttggg-3′ | (SEQ ID No 17) |

The kappa light chain variable domain primers which start the amplification at the COOH terminus are:—

| | |
|---|---|
| Vκ1a | |
| 5′-gacatcgagctcacccagtctcca-3′; | (SEQ ID No 18) |
| Vκ1s: | |
| 5′-gacatcgagctcacccagtctcca-3′; | (SEQ ID No 5) |
| Vκ2a: | |
| 5′-gatattgagctcactcagtctcca-3′; | (SEQ ID No 6) |
| Vκ3a | |
| 5′-gaaattgagctcacgcagtctcca-3′; | (SEQ ID No 19) |
| and | |
| Vκ3b: | |
| 5′-gaaattgagctcacg(g/a)cagtctcca-3′. | (SEQ ID No 7) |

The above light chain variable region primers were used in a PCR reaction with a light chain constant domain primer:—

| | |
|---|---|
| CK1d | |
| 5′-gcgccgtctagaattaacactctcccctgttga | (SEQ ID No 20) |
| agctctttgtgacgggcgaactcag-3′. | |

Owing to the wide variety of variable region primers that could be used, all primers were used in all amplifications.

The RT-PCR mix freshly prepared from the reverse transcription reaction of the RNA described above consisted of 792 μl DNAse free water, 100 μl (10×) Taq buffer, and 8 μl dNTP's (DATP, dCTP, dGTP, and dTTP each at 25 mM). 90 μl of this PCR mix were added to a new PCR reaction tube. 3 μl of 5′ primer, and 3 μl of 3′ primer, i.e. 60 pmoles of each primer (each at 20 μM) were then added to each PCR reaction tube.

5 Units (0.5 μl) of Taq polymerase was then added to each reaction tube. 2 μl of cDNA (originally containing 2 μl of RNA) was then added. Two drops of mineral oil were then added to the top of each reaction tube, and 25-40 rounds of PCR amplification were then carried out. PCR reaction details were as follows:—

40 cycles of 94° C. for 30 seconds, 52° C. for 50 seconds, and 68° C. for 90 seconds. 10 μl of the PCR reaction product were then removed, and 2 μl of 6× loading buffer was then added. The resultant mix was run on a 2% agarose gel (50:50 of normal:low melting point agarose) with Phi 174/Hae III marker. A strong band of approximately 660 bp was indicative of a successful PCR amplification of DNA encoding the heavy chain variable (VH) and heavy chain constant (CH1) regions, or the light chain variable (VL) and light chain constant (CL) regions.

The heavy chain and light chain PCR products were gel-purified, extracted (Wizard® PCR Preps DNA Purification System, Promega, UK) and reamplified using extension primers with a 5′ poly (GA) tail to increase restriction enzyme digestion and cloning efficiency [Williamson, 1993 Williamson R A, Burioni R, Sanna P P, Partridge L J, Barbas C F, 3rd, Burton D R. Human monoclonal antibodies against a plethora of viral pathogens from single combinatorial libraries Proc Natl Acad Sci USA 90, 4141-5, 1993]. The PCR reactions were as follows:—

40 cycles of 94° C. for 30 seconds, 52° C. for 50 seconds, and 68° C. for 90 seconds.

Initial phage library construction used the pComB3HSS phage display vector which is illustrated in FIG. 1 and was a gift from the Scripps Research Institute, La Jolla, USA. The concentration of PCR amplified heavy and light chain DNA for restriction digestion was first determined. The light chain PCR fragment (VL and CL) was then digested with SacI/XbaI, and the heavy chain PCR fragment (VH and CH-1) was digested with SpeI/XhoI (GibcoBRL). The digests were then sequentially ligated into pComB3HSS vector which had been pre-cut with the same enzymes as that for the PCR fragment being ligated therein, i.e. usually the light chain was cloned into the vector first, followed by the heavy chain.

1400 ng of suitably digested vector was added to a reaction tube with 450 ng of suitably digested PCR product, i.e. either a heavy chain fragment (VH and CH-1) or light chain fragment (VL and CL), 40 μl of 5× ligase buffer and 10 μl of ligase in a total volume of 200 μl. The ligation was incubated at room temperature overnight, and then heat killed for 10 min at 70° C. The DNA was precipitated and the resultant DNA pellet drained and rinsed with 70% ethanol and then allowed to dry on a paper towel. The pellet was dried further on a Speedvac and resuspended in 15 μl water. The tube was placed on ice for 10 min. Positive control ligations were carried out to confirm ligation had worked.

Electrocompetent E. coli XL-1Blue cells (300 μl per ligation) were then thawed and added to a tube containing ligated vector DNA, mixed and set for 1 minute. The cell/DNA mix was transferred to an electroporation cuvette, and electroporation was carried out as follows.

To electroporate, the cells were pulsed at 2.5 kV, 0.2 cm gap cuvette, 25 μFD and 200%. The electroporation cuvette was flushed immediately, first with 1 ml, and then with 2 ml of SOC medium at room temperature, followed by immediate incubation in this SOC medium for 1 hr at 37° C. in a shaker (250 rpm). 10 ml of prewarmed (37° C.) Superbroth (SB) containing 20 μg/ml carbenicillin and 10 μg/ml tetracyclin was then added. Transformants were immediately titred by plating 100 ml, 10 ml, and 1 ml for the control test ligation, and 10 ml, 1 ml, and 0.1 ml for library ligation on LB plates containing 100 μg/ml carbenicillin.

The 10 ml culture of transformed E. coli was incubated for 1 hr at 37° C. on a shaker (300 rpm). Following incubation, carbenicillin was added to a final concentration of 50 μg/ml and incubated for an additional hour at 37 μC. 10 ml of the culture were added to 100 ml of SB containing 50 μg/ml carbenicillin and 10 μg/ml tetracycline, and incubated overnight at 37° C. on a shaker.

Following overnight growth, phage plasmid DNA was isolated by miniprepping. The isolated vector was checked for insertion of the first chain and prepared for ligation with the next chain. Usually the light chain was cloned into the vector first, followed by the heavy chain. Hence, the resultant phage library consisted of a combinatorial phage having both the light and the heavy chain inserted therein.

Biopanning

Biopanning was carried out to isolate Fab-phages which expressed anti-platelet antigen specific immunoglobulins as follows.

Platelets were prepared by centrifugation of 60 ml anti-coagulated blood at 200 g for 10 min at room temperature. An ELISA plate was then coated with 50 μl of a platelet suspension equivalent to 108 platelets in bicarbonate buffer pH 8.6, and incubated overnight at 4° C. The following morning, after washing twice with TBS (Tris Buffered Saline), the plate was blocked with 5% BSA (Bovine Serum Albumin) in PBS (Phosphate Buffered Saline) and incubated for one hour at 37° C. After two further washes with TBS, 100 μl phage suspension was added to each well and the plate incubated for two hours at 37° C. Isolation of the phages is described below.

The phage was then eluted with 50 μl of elution buffer per well, and was incubated with TBS 0.05% Tween 20 (TBST) at room temperature for 10 minutes. After vigorously pippetting, the eluted phage was removed and washed by neutralisation with 3 μl of 2M Tris base. For the first round of panning, the plate was washed by this method once. In the second round of panning, five washes were used and in the third and subsequent rounds, ten washes were used.

Referring to FIG. 2, there is shown the results of carrying out five rounds of biopanning. Following each round of panning, the number of eluted anti-platelet antigen specific Fab-phage was determined by titration on Luria Broth (LB) containing carbincillin (100 μgml⁻¹) and the number of colony forming units were counted (cfu/ml) and indicated as mean (±SE).

As can be seen from FIG. 2, an enrichment of anti-platelet antigen specific Fab-phage was achieved by the biopanning, as a culture of the first round of panning only showed that only two anti-platelet antigen specific Fab-phage clones were isolated from 10⁹ cfu/ml phage suspension, whereas after the fifth round of panning, approximately 70 clones were isolated from 10⁹ cfu/ml phage suspension.

Determination of the Phage's Specificity for Platelets

The pool of anti-platelet antigen specific Fab-phage obtained after the five rounds of panning as illustrated in FIG. 2, was analysed by ELISA to determine the anti-platelet antigen specificity of the Fab fragment produced by the phage. Platelets were prepared by centrifugation of 60 ml anti-coagulated blood at 200 g for 10 min at room temperature.

A microtitre plate was coated with 50 μl platelet concentrate equivalent to 10⁸ platelets, then sealed and incubated at 4° C. overnight. The plate was washed twice with PBS, blocked with 5% BSA in PBS and placed at 37° C. for one hour. After shaking out the blocking solution, 100 μl phage containing 10⁸ pfu was added to each well. 100 μl M13 phage containing same concentration was added as a negative control. After washing six times with PBS/Tween 20, 100 μl rabbit anti phage antibody (7 μg ml⁻¹ Sigma Co.) was added to all the wells except the blank and incubated for one hour at 37° C. The plate was washed with wash buffer (0.05% Tween 20 in TBS) six times. 100 μl specific anti-rabbit horseradish peroxidase conjugated antibody (1:10,000 diluted in PBS/1% BSA) was added to each well and incubated at 37° C. for one hour. After washing six times with washing buffer, 200 μl of substrate buffer (10 μg ml⁻¹ OPD in citrate buffer) was added and the plate was left in the dark for 30 minutes. The reaction was stopped by adding 25 μl of 3M HCL, and finally read by an ELISA reader at 490 nm.

Referring to FIG. 3, there is shown the platelet membrane binding activity of six representative clones (S1-S6) selected from the anti-platelet antigen specific Fab-phage library by five rounds of biopanning, including an M13 phage negative control (N) and a blank well (B). The data represents the mean absorbance ±SE from two experiments and, in each experiment, three wells were used for every determination.

FIG. 3 shows that out of the six randomly selected Fab-phage clones of different colony size, four clones (S1,S2,S3,S4) reacted strongly with a whole platelet preparation, whereas two clones (S5,S6) failed to react with platelet antigens. No reaction was seen with the negative and blank controls.

The Reaction of Platelet Reactive Phage Against a Platelet Lysate Preparation

The four Fab-expressing phages (S1-S4) that showed a positive reaction against whole platelets as shown in FIG. 3, were analysed by ELISA to detect anti-platelet antigen specificity against platelet lysate. The method used was exactly the same as for the whole platelet preparation described above, except that 50 μl of platelet lysate (200 gml⁻¹) was added to the microtitre plate prior to washing with PBS and addition of 100 μl phage. Platelet lysate was produced as follows.

60 ml anti-coagulated blood was centrifuged at 200 g for 10 minutes at room temperature. The platelet rich plasma (PRP) was removed and re-centrifuged by at 1200 g for 10 min (Mistral 3000, Fisons Ltd, UK). After washing, the sedimented platelets four times with isotonic buffer, 6 ml of lysis buffer was added, and incubated at 4° C. for one hour, after which it was centrifuged in an Avanti™ J-25 centrifuge (Beckman) at 20,000 g for 30 minutes at 4° C. 5 ml of the supernatant was concentrated using centrifugal filter device (10,000 MW Cut Off) at 4000 g for 40 minutes.

Referring to FIG. 4, it is shown that the reactivity of Fab-expressing phage against platelet lysate, including an M13 phage negative control (N) and a blank well (B). Results show that all four selected clones (S1-S4) which reacted strongly with platelet surface membrane antigens in whole platelet suspension as shown in FIG. 3, also reacted strongly with platelet lysate, when compared with the negative control and the blank.

Sensitivity of Phage Binding to Platelet Membrane Proteins

100 μl of platelet lysate was used to coat each well of a Maxisorp microtitre plate. The suspension containing 106 reactive anti-platelet antigen specific Fab-phage was diluted over the range of 1×10⁻¹ to 1×10⁻⁶ cfu, and 100 μl of each dilution was added to each well. The binding of phage was detected with conjugated anti-phage antibody.

Referring to FIG. 5, there is shown a titration of Fab-bearing phage against a platelet lysate preparation (Mean ±SE). FIG. 5 shows that the original concentration of 1×10⁶ cfu of Fab phage gave the highest absorbance. Decreasing absorbance correlated with decreasing concentration of Fab-bearing phage.

The Reaction of Phage Particles Against Purified Platelet Glycoprotein IIb/IIIa

To demonstrate that the anti-platelet antigen specific Fab-phage particles specifically react with glycoprotein IIb/IIIa, they were tested against purified protein by ELISA. Therefore, those colonies which showed a strong reaction with both whole platelets (see FIG. 3) and also platelet lysate (see FIG. 4), were analysed to determine their reactivity against purified glycoprotein IIb/IIIa.

ELISA was carried out as previously for whole platelets as shown in FIG. 3, and platelet lysate as shown in FIG. 4. Each well of an ELISA microplate was coated with 100 μl of pure glycoprotein IIb/IIIa at a concentration of 2 μg/ml. The glycoprotein IIb/IIIa was a gift from Dr Beat Steiner, University Basel, Switzerland Following washing with PBS, 100 μl of phage was then added to each well and anti-phage antibody was used to detect their specificity for glycoprotein IIb/IIIa.

Referring to FIG. 6, there is shown the reactivity of phage against purified platelet glycoprotein IIb/IIIa. The results indicate that all four selected clones (S1-S4) which reacted strongly with platelet antigens in whole platelet suspension and platelet lysate, also strongly reacted with purified glycoprotein IIb/IIIa complex, when compared with an M13 negative control (N) and blank (B).

Western Blotting—with Fab Anti-Platelet Antibodies Library Against Platelet Membrane Lysate

Western blotting was carried out with the Fab-expressing phage clones isolated previously described (S1-S4) as follows.

A platelet lysate sample was prepared by diluting one part sample buffer to three-parts platelet lysate (200 (gml⁻¹) and boiling for two minutes. An 8% resolving gel was poured and allowed to set, after which a 3% stacking gel was added. 40 μl of platelet lysate sample and 5 μl molecular weight (MW) marker, was added to separate wells in the gel. The gel was run at 110V constant voltage for 2 hours. The protein was transferred from polyacrylamide gel to 0.45 μm nitrocellulose membrane and was run at 100V for 90 min. Nitrocellulose membrane strips, containing the separated platelet proteins, were blocked with 5% BSA in TBS for 2 hours at room temperature. 3 ml of eluted phage 108 cfu/ml was placed onto the separate sample strips. 3 ml of M13 at a same concentration acted as a negative control, and 3 ml anti GPIIb/IIIa antibody (2 μg ml⁻¹) as the positive control. All samples and controls were incubated at 4° C. overnight.

The strips were washed with TBS/0.05% Tween 20 for two hours; every 20 minutes the wash buffer was changed. 3 ml rabbit anti-phage antibody diluted 1:1000 was added to all samples (except the positive control) and incubated at room temperature for one hour.

After washing as before, 3 ml of anti-rabbit horseradish peroxidase conjugated antibody diluted 1:10,000 was added to all samples and negative controls strips.

For the positive control, 3 ml of anti-mouse horseradish peroxidase conjugated antibody was added at the same dilution. After incubation at room temperature for 1 hour, the strips were washed as previously. The blot was developed with 3 ml of Diaminobenzedine.

Referring to FIG. 7, there is shown a Western blot of non-reduced human platelet membrane lysate binding with the four isolated anti-platelet Fab-bearing phage clones (S1-S4). Lane N is an M13 phage negative control, and lane P is a positive control stained with an antibody against CD61, i.e. the β integrin component of the IIa/IIIb complex. A Molecular weight marker was run in the MW lane.

Lanes S1-S4 indicate the four isolated Fab phages (S1-S4) bind with platelet protein bands having molecular weights of 11 KDa and 92 KDa, respectively.

Western Blotting—with Library Fab Anti-Platelet Antibodies Against Pure GPIIb/IIIa

In order to confirm that the isolated Fab-expressing phage library colonies specifically bind with the platelet glycoprotein GPIIb/IIIa, 40 μl of pure platelet glycoprotein IIb/IIIa at a concentration of 8 μg/ml was loaded on to a polyacrylamide gel, electrophoresed and transferred on nitrocellulose paper. 10⁶ Fab library phage was added to each sample strip. Rabbit anti-phage HRP conjugated antibody was then added and the blot developed as described above.

Referring to FIG. 8, there is shown the result of western blotting of non-reduced human platelet GPIIb/IIIa glycoprotein against the four platelet reactive phage clones together with an M13 phage negative control (N) and a CD61 positive control (P). A molecular marker was also run in the MW lane.

Lanes S1-S4 indicate that the four Fab anti-platelet antibodies all bind with GPIIb/IIIa, with molecular weight of 92 KD.

Conversion of Pcomb3 from Phage Display to Soluble Fab

The data previously presented demonstrates that the Fab-expressing phages bind to the platelet antigen, and does not formally show that the Fab molecule expressed by the phage specifically recognises, and immunoreacts with GPIIb/IIIb. This is because the Fab molecule is formed as a chimeric protein with a phage coat protein which is expressed and attached to the surface of the phage.

In order to determine whether the Fab phage clones specifically recognise and react with GP IIb/IIIa, the phage coat protein was removed, and the Fab expressed as a soluble protein unattached to the phage. Soluble Fab fragments were prepared from four colonies that reacted strongly with platelet antigens as described below.

5 μgml⁻¹ of phage DNA containing heavy and light chain inserts was digested for 3 hours at 37° C. with 2 μg of the restriction enzymes Spe1 and Nhe1 (10 Upl⁻¹) to remove the DNA encoding the PIII coat protein. The cut DNA was then loaded on to a 0.6% low melt agarose gel and electrophosesed at 4° C. The 4.7 Kbp band of phage containing the heavy and light chains but not the DNA encoding the PIII coat protein was cut from the gel and recovered using the Gene Clean system (Promega).

200 ng of the 4.7 Kbp recovered DNA was ligated with 2 μl of ligase (2 Upl⁻¹) in a 20 μl total volume of ligase buffer for 2 hours at room temperature. 1 μl of the ligated DNA was added to 40 μl of competent E. coli cells and transfected by electroporation pulsing at 2.5 KV, 25 μFD and 200Ω.

The electroporated cells were transferred to 10 ml of superbroth containing 20 μgml⁻¹ carbenicillin and 10 μgml⁻¹ tetracycline. Immediately the cells were inoculated on to LB plates containing 100 μgml⁻¹ carbenicillin. After 24 hours, a single colony was inoculated into 10 ml of superbroth containing 20 mM MgCl₂ and 50 μgml⁻¹ carbenicillin and incubated at 37° C. for 6 hours. Expression of the protein was induced by adding Isopropyl β-D thiogalacotsipyranosid to a final concentration of 1 mM. The cells were recovered by centrifugation for 15 minutes at 1500 g and the soluble Fab was recovered from the cell lysate.

The reactivity of soluble Fab fragments with platelet in the cell lysate supernatant was determined by ELISA. Referring to FIG. 9, there are shown the results for three (S1, S3, S4) of the four reactive phage colonies. The fourth (S2) clone appeared not to express soluble phage. All three soluble Fab molecules show significant reactivity against platelet membrane proteins.

DNA Sequence Analysis

The Fab molecule of the S4 isolate was sequenced by preparing plasmid DNA using a High Pure Plasmid Isolation Kit. Nucleic acid sequencing was carried out by Cambridge Bio-sciences, using the primers:—

| | |
|---|---|
| 5′-gaaatacctattgcctacgg-3′ | (SEQ ID No 8) |

for the heavy chain of the antibody; and

| | |
|---|---|
| 5′-gcgattgcagtggcactgg-3′ | (SEQ ID No 9) |

for the light chain of the antibody.

The variable region genes used were attributed by using the V-Base program available at:—

http://www.mrc-cpe.cam.ac.uk/vbase-ok.php?menu=901

The DNA sequence of the immunoglobulin heavy chain variable region (VH) is illustrated as SEQ ID No 10. The DNA sequence of the immunoglobulin light chain variable region (VL) is illustrated as SEQ ID No 12.

DNA sequences of the heavy and light chains were translated on-line (www.expasy.org/tools/dna.html). The amino acid sequence of the immunoglobulin heavy chain is illustrated as SEQ ID No 11. The amino acid sequence of the immunoglobulin light chain is illustrated as SEQ ID No 13.

Alignment of the amino acid sequences of the heavy and light chains with germline sequences was carried out using DNAPLOT and VBASE.

Referring to FIG. 10, there is shown a sequence alignment comparing the amino acid sequence of the heavy chain of the expressed antibody with the germline gene from which the antibody is derived. Three Complementary Determining Regions, VH-CDR1, VH-CDR2 and VH-CDR3 of the heavy chain are shown underlined. The V-base program indicated that the heavy chain is generated by the variable region genes DP 58, D-7-27 and JH4b. The dots means sequence identity with the germline genes.

Referring to FIG. 11, there is shown a sequence alignment comparing the amino acid sequence of the light chain of the expressed antibody with the germline genes (DPK18/A17+) from which the antibody is derived. Three Complementary Determining Regions, VL-CDR1, VL-CDR2 and VL-CDR3 of the light chain are shown. The V-base program indicated that the light chain uses the variable region genes DPK 18 and JK 2.

The results indicate that there is approximately 80% homology between the DNA encoding the Fab region expressed by the phage and the germline genes from which it is derived. Furthermore, the results indicate that because the mutations occurred in CDRs, antigen drive has played a significant role in the development of this antibody.

The mutation from Tyrosine (Y) to Arginine (R) at residue position 31 of the light chain creates an RSD motif which shows a similarity with the RGD motif which is a major integrin ligand. RSD has not been previously described as an integrin ligand.

Peptide ligand sites for a number of integrins have been described in Plow et al. (J. Biol. Chem. Vol. 275 (29) 21785-21788). Therefore, it is possible to search for putative ligand mimitopes (sites that appear to mimic the ligand) within the antibody sequence.

Referring to FIG. 12, there are shown two potential mimitopes found in the VL-CDR1 of the light chain. One is the putative mimic of the RGD motif described as binding to a number integrins, i.e. RSD. There is also some identity with the Tenascin binding motif AEIDGIEL and it would be simple to introduce this motif by mutation into the structure of the antibody. In addition, there is also a reverse of the GPR sequence but this is in the framework region of the immunoglobulin and may not be accessible. Inhibitory motifs do not have to contain homologues of RGD and many studies have shown peptides containing RGD to be activating (Smith et al.). Thus, the presence of this motif is not predictive of function and there are aspects of the region described by Smith et. al. that are not to be found in this sequence.

### Fab-Expressing Phage Binding to Platelet Antigens Determined by Flow Cytometry

Washed whole platelets were reacted with the four phage colonies S1-S4 and binding was determined by a fluorescent anti-phage complex as follows.

100 μl containing 10⁷ washed platelets were incubated for 1 hour at 37° C. with 100 μl of 10⁷ Fab-phage. For a negative control, 100 μl of the same concentration of platelets were incubated with the same concentration of M13. The tubes were washed with wash buffer and centrifuged for five minutes at 1200 g.

Bound phage was detected with 200 μl of rabbit anti-phage antibody at a concentration of 1:200 dilution of 35 μg/ml at 37° C. for 30 min. After washing twice with wash buffer, 200 μl of anti-rabbit FITC conjugated antibody at 1:2000 dilution of an original 20 μgml⁻¹ concentration was added to the samples and the negative control tube. 5 μl of anti-GPIIb FITC conjugated antibody was added to the positive control tube and incubated for 30 min at room temperature in the dark.

The samples and controls were analysed by a Flow-cytometer at the following thresholds: Forward Scatter Side (FSC): E01, Side Scatter (SSC): 575 and Fluorescence (FL1):400 (Becton Dickinson).

Referring to FIG. 13, there is shown the results of Flow Cytometric analysis of platelet antigen binding activity of the four representative clones (S1-S4) from the Fab phage library. Data represents the mean percentage of fluorescence ±SE from four experiments. Positive control (anti-GPIIb) was used to detect the gate that was used to determine platelet fluoresence. In addition, FIG. 13 shows positive (Con), negative (M13 phage) and blank controls (Neg). As shown in FIG. 13, all four clones bind to between 40-60%, of the platelet population

These results provide further support that the isolated phage bind to platelets as previously suggested by ELISA and western blotting analyses.

### Flow Cytometric Binding of Fibrinogen to Platelet in Presence of Isolated Fab Anti-Platelet Antibody

Platelet rich plasma (PRP) was incubated with the four isolated phage clones to determine whether these antibodies blocked fibrinogen binding to resting platelets, and platelets activated by differing concentrations of ADP, as described below.

5 μl of normal platelet rich plasma (PRP) were incubated with 10 μl of isolated Fab-phagemid (1.5×10⁸ cfu) for one hour at 37° C. 10 μl of same concentration of M-13 phage and 10 μl of TBS buffer and 10 μl Tyrode's buffer were added to appropriate tubes as controls and a further 20 μl of Tyrode's buffer was added to all tubes.

2 μl of anti-fibrinogen antibody Fluoroscein Isothiocyanate (FITC) was added to each of the samples and controls, Spl of different concentrations (0.1, 1, 10 (mol ml⁻¹) of ADP were then added to the approriate tubes. 5 μl of anti-IIb conjugated FITC was added to PRP for determination of the positive platelet gate. The volume of all samples was adjusted to 50 μl by adding Tyrode's buffer. All samples were left in darkness for 30 minutes.

The samples were fixed with 450 μl of 1% paraformaldehyde and examined on FACScan, at the following threshold: Forward Scatter Side (FSC): E01, Side Scatter (SSC): 575 and Fluorescence (FL1): 400 (Becton Dickinson).

Referring to FIG. 14, there is shown a summary of the percentage of the mean fluorescence intensity (MFI) reflecting the binding of fibrinogen to platelets incubated with either one of the four phage preparations (S1-S4), an M13 phage preparation as negative control, and buffer, each of which was exposed to different concentrations of ADP (0.1, 1, 10 μmol/ml).

It can be seen that, whilst the controls show an increase in the binding of fibrinogen to platelets as the concentration of ADP is increased, no increase is seen in platelets incubated with clones S1-S4. These results indicate that the phage bearing the Fab is reactive with platelet GP IIb/IIIa and can inhibit the binding of fibrinogen to platelets activated by ADP. The binding of fibrinogen to the platelet to GP IIb/IIIa is the first and crucial event in the aggregation of platelets and therefore in the commencement of the formation of a clot. It is thus a prime target for therapy designed to prevent clots.

The reader's attention is directed to all papers and documents which are filed concurrently with or previous to this specification in connection with this application and which are open to public inspection with this specification, and the contents of all such papers and documents are incorporated herein in reference.

All of the features disclosed in this specification (including any accompanying claims, abstract and drawings, and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A recombinant human immunoglobulin or functional fragment thereof comprising at least one antigen binding region having an amino acid sequence comprising:
(i) residues 31-35 of SEQ. ID No:11;
(ii) residues 50-66 of SEQ. ID No:11;
(iii) residues 100-110 of SEQ. ID No:11;
(iv) residues 24-40 of SEQ. ID No:13;
(v) residues 56-61 of SEQ. ID No:13; and
(vi) residues 95-102 of SEQ. ID No:13.

2. A recombinant human immunoglobulin or functional fragment thereof comprising at least one antigen binding region encoded by a polynucleotide having a nucleotide sequence comprising:
(i) residues 91-105 of SEQ. ID No:10;
(ii) residues 148-198 of SEQ. ID No:10;
(iii) residues 298-330 of SEQ. ID No:10;
(iv) residues 70-120 of SEQ. ID No:12;
(v) residues 166-183 of SEQ. ID No:12; and
(vi) residues 283-306 of SEQ. ID No:12.

3. A recombinant human immunoglobulin or functional fragment thereof comprising a light chain variable region (VL) and/or a heavy chain variable region (VH), the light chain variable region having an amino acid sequence which is substantially as set out in SEQ ID. No. 13, the heavy chain variable region having the amino acid sequence which is substantially as set out in SEQ ID. No. 11.

4. A recombinant human immunoglobulin or functional fragment thereof comprising a light chain variable region (VL) and/or a heavy chain variable region (VH), the light chain variable region being encoded by a polynucleotide having a nucleotide sequence which is substantially as set out in SEQ ID. No:12, the heavy chain variable region being encoded by a polynucleotide having a nucleotide sequence which is substantially as set out in SEQ ID. No:10.

5. An isolated peptide comprising an amino acid sequence comprising:
(i) residues 31-35 of SEQ. ID No:11;
(ii) residues 50-66 of SEQ. ID No:11;
(iii) residues 100-110 of SEQ. ID No:11;
(iv) residues 24-40 of SEQ. ID No:13;
(v) residues 56-61 of SEQ. ID No:13; and
(vi) residues 95-102 of SEQ. ID No:13.

6. (canceled)

7. An isolated polynucleotide comprising a nucleotide sequence selected from the group consisting of:
(i) residues 91-105 of SEQ. ID No:10;
(ii) residues 148-198 of SEQ. ID No:10;
(iii) residues 298-330 of SEQ. ID No:10;
(iv) residues 70-120 of SEQ. ID No:12;
(v) residues 166-183 of SEQ. ID No:12; and
(vi) residues 283-306 of SEQ. ID No:12.

8-12. (canceled)

13. A method of treating blood clotting disorders and/or inflammation, the method comprising administering to a patient a recombinant immunoglobulin or functional fragment thereof according to any of claims 1 and 3, a peptide according to claim 5, or a polynucleotide according to claim 7, each being optionally derivatised.

14. A pharmaceutical composition comprising a recombinant immunoglobulin or functional fragment according to any of claims 1 and 3, a peptide according to claim 5, or a polynucleotide according to either claim 7, each being optionally derivatised, and a pharmaceutically acceptable excipient, carrier, buffer or stabiliser.

15-35. (canceled)

36. A recombinant DNA molecule comprising a polynucleotide according to claim 7, or derivative thereof.

37. A cell containing a recombinant DNA molecule according to claim 36.

38. (canceled)

39. A method of isolating a recombinant immunoglobulin or functional fragment thereof having ability to bind to a target integrin, the method comprising:
(i) mutating a recombinant immunoglobulin or functional fragment thereof according to any of clams 1 to produce a mutant, and
(ii) selecting the mutant for activity against a target integrin.

40. The method of claim 39, wherein said mutating comprises random mutagenesis.

41. The method of claim 39, wherein said mutating comprises introducing at least one ligand having immunospecificity against a target integrin into at least one antigen binding region of the recombinant immunoglobulin or functional fragment thereof.

42. The method of claim 41, wherein the at least one ligand is independently selected from a group of ligands consisting of RDG, RSG, RSD, HHLGGAKQAGDV, GPR, RPG, AEIDGIEL, ARSDGVSLM, QIDS, LDT, IDAPS, DLXXL, and GFOGER.

43-47. (canceled)

48. A recombinant human immunoglobulin or functional fragment thereof according to any of claims 1 or 3, wherein at least a part of the antigen binding region of the immunoglobulin or fragment comprises a ligand having immunospecificity to a desired target integrin.

49. The recombinant human immunoglobulin or functional fragment thereof according to claim 48, wherein the ligand is selected from the group consisting of RDG, RSG, RSD, HHLGGAKQAGDV, GPR, RPG, AEIDGIEL, ARSDGVSLM, QIDS, LDT, IDAPS, DLXXL, and GFOGER.
